(19) United States
(12) Patent Application Publication
(10) Pub. No.: US 2025/0164477 A1
(43) Pub. Date: May 22, 2025

(54) Single Waveform, Continuous Squarewave Voltammetry for Optimal Calibration Free Sensing

(71) Applicant: University of Cincinnati, Cincinnati, OH (US)

(72) Inventors: Sanduni Wasuththara Abeykoon, Cincinnati, OH (US); Jordan Michelle Seibold, Cincinnati, OH (US); Lars Alexander Olivan, Cincinnati, OH (US); Ryan White, Cincinnati, OH (US)

(73) Assignee: University of Cincinnati, Cincinnati, OH (US)

(21) Appl. No.: 18/839,249

(22) PCT Filed: Feb. 21, 2023
(86) PCT No.: PCT/US2023/013521

§ 371 Date: Aug. 16, 2024

Related U.S. Application Data
(60) Provisional application No. 63/311,704, filed on Feb. 18, 2022.
(60) Provisional application No. 63/353,172, filed on Jun. 17, 2022.

(51) Int. Cl. G01N 33/543; G01N 33/53

(52) U.S. Cl. CPC G01N 33/5438; G01N 33/5308

(57) ABSTRACT

A method of measuring sensor response for a sample is disclosed. The sample is exposed to an electrochemical aptamer-based (EAB) sensor, where the sensor includes an electrode and one or more aptamers having redox tags. Next, an interrogation is performed by applying an abrupt voltage pulse to the electrode. Two or more data samples are collected at different time values, where each data sample is a redox tag current value. Then at least one measure of the sample is identified using the data samples.

### CROSS-REFERENCE TO RELATED APPLICATIONS

This application is a continuation of PCT Application No. PCT/US23/13521 filed Feb. 21, 2023, which claims the benefit of the filing date of U.S. Provisional Application No. 63/311,704 filed Feb. 18, 2022, and U.S. Provisional Application No. 63/353,172 filed Jun. 17, 2022, the disclosures of which are incorporated by reference herein in their entirety.

### FIELD OF THE INVENTION

This invention relates generally to square wave voltammetry.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Square wave voltammetry (SWV) possesses several unique characteristics that have enabled the technique to become a mainstay in measuring surface bound redox systems. These characteristics result from the pulsed potential waveform and coordinated current measurement that precludes interferences from non-faradaic processes. Nominally, the technique utilizes a square wave potential pulse waveform that is superimposed on a staircase step to generate a voltammetric linear sweep. The current sampling technique of SWV typically only considers the current response during the latter half of the potential pulse, which minimizes any non-faradaic contributions to the resulting current response. The current-voltage curve is presented as the difference between the forward and reverse current samples (taken on at the forward and reverse pulse), which further reduces any remaining charging current and amplifies the peak current providing square wave voltammetric data with high sensitivity.

SWV is useful for measuring surface-bound processes. It facilitates the monitoring of current responses as a function of frequency and allows for characterization of the charge transfer rates in these systems. It has been demonstrated that SWV peak currents can be used to determine the standard rate constant by finding the critical frequency, which is the maximum of the relationship between normalized peak current and inverse frequency. The normalized peak current is defined as the ratio between peak current (iₚ) and frequency (f). The normalized current maximum occurs in an analogous way to resonance frequency in spectroscopy—current is maximized when the perturbation frequency, and thus characteristic time scale of the voltammetric experiment, is similar to that of the charge transfer rate. Prior work utilized SWV to determine the kinetic parameter, defined as the standard rate constant and frequency ratio. This type of critical frequency analysis was similarly applied to folding based sensors that utilized functional nucleic acids, and the target induced conformation change, to build sensors. It was demonstrated that the critical frequency, and thus the charge transfer rate, changes upon target addition which results in a conformation change of the surface-bound nucleic acids.

The power of SWV, as described above, relies on its ability to sample current at the end of each forward and reverse potential pulse (defined as forward current i_{f} and reverse current iᵣ; FIG. 1A). The delay in current sampling allows non-faradaic processes to decay before sampling current contributions from faradaic processes occurring at the electrode surface. Schematically, the current is sampled at the end of the forward and reverse pulse, but commercial instruments sample several current points along the potential pulse and average these data for better signal-to-noise ratio voltammograms. While the exact region of the pulse that is collected and utilized for data analysis may vary between instrument manufactures, the net result is averaged current for better signal and current is discarded at the beginning of the pulse. While this works well for many applications, as demonstrated in the field of electrochemical aptamer-based sensors, this method leaves data behind. Therefore, a need still exists for a more efficient method of collecting data for square wave voltammetry.

SWV, and chronoamperometry, and other fundamental approaches for measuring measuring EAB sensors also fundamentally contain additional information that can allow for sensor calibration. Each technique is a specific exploitation of fundamental electron transfer rates in EAB sensors. These calibration techniques have their drawbacks including complexity of measurement waveforms and limits on the amount of information collected for analysis and improved calibration. Therefore, a need still exists for a more efficient method of collecting data for sensor calibration and ideally also doing so in a manner that can also collect more information for improved calibration.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes. In one aspect of the present invention, a method of measuring sensor response for a sample is disclosed. The method involves exposing the sample to an electrochemical aptamer-based (EAB) sensor, where the sensor comprises an electrode and one or more aptamers having redox tags. Next, performing a first interrogation comprising applying an abrupt voltage pulse to the electrode, where the abrupt voltage pulse causes redox electron transfer between the redox tags on the aptamers and the electrode. Then, collecting two or more data samples at different time values, where each data sample is a redox tag current value. Finally, identifying at least one measure of the sample using the data samples.

In one embodiment, square wave voltammetry is used to interrogate the sensor. In another embodiment, amperometry is used to interrogate the sensor. In one embodiment, electron transfer rate data is collected after a time period selected from the group consisting of 10 μs, 100 μs, and 1 mS. In another embodiment, electron transfer rate data is collected after at least one time period selected from the group consisting of 10 μs, 100 μs, and 1 mS. In one embodiment, sampling is performed at a frequency that is selected from the group consisting of at least 10 Hz, 100 Hz, 1 kHz, 10 kHz and 100 kHz.

In another embodiment, interrogating is conducted without calibration of the sensor. In one embodiment, the sensor has been calibrated. In another embodiment, calibration is conducted during manufacture of the sensor. In one embodiment, calibration of the sensor is performed before use of the sensor. In another embodiment, calibration of the sensor is performed during use of the sensor. In one embodiment, the interrogating is conducted twice, wherein one interrogation is done with a target and another interrogation has no target.

In another embodiment, the two or more samples comprise a minimally responsive sample wherein redox tag current is minimally responsive to a change in concentration of a target, and a responsive sample which has a response to a change in concentration of the target. Current measurements of the two or more samples are used to calibrate the sensor. In one embodiment, calibration of the sensor is calculated, at least in part, by comparing current measurements of the minimally responsive sample and the responsive sample at the time of response at saturation level through a polynomial fit.

In another embodiment, the calibration provides a concentration value, the concentration value calculated according to

$$[ T ] = {K}_{D} \frac{i - α {i}_{NR}}{γα {i}_{NR} - i} = {K}_{D} \frac{\frac{i}{{i}_{NR}} - α}{γα - \frac{i}{{i}_{NR}}} \qquad Equation 1$$

wherein [T] is the concentration of the target, K_{D} is the target's dissociation constant, i is a constant comprising the peak current, α is a constant comprising the ratio of output signal at the minimally frequency and target-free output signal, i_{NR} is output current, and γ is a constant comprising the ratio of target-saturated output signal to target-free output signal.

In one embodiment, the two or more samples comprise a first sample and a second sample, and wherein the first sample has a redox tag current that increases with increase in concentration in target and the second sample has a redox tag current that decreases with increase in concentration in target, and the difference in changes in current between these two samples is recorded as a differential current value. In another embodiment, the two or more samples are an average sample averaged from two or more adjacent samples. In one embodiment, the two or more samples are a composite sample obtained from two or more adjacent samples.

In another embodiment, at least one sample is taken at a time point equivalent to which the change in redox tag current has the largest increase for a given increase in target concentration. In one embodiment, at least one sample is taken at a time point equivalent to which the change in redox tag current has the largest decrease for a given increase in target concentration. In another embodiment, at least one sample is taken at a time point equivalent to which the change in redox tag current has least change for a given increase in target concentration. In one embodiment, the method is a continuous square wave voltammetry scan, and the sensor is measured over time using at least one continuous square wave voltammetry scan for calibration and a plurality of non-continuous square wave voltammatery scans for measurement. In another embodiment, the sensor is calibrated during in-vivo use at known points where concentration of the target analyte is less than a percentage selected from the group consisting of 2, 5, 10 and 20%. In one embodiment, the data samples are used to determine critical frequency.

In another embodiment, the applied abrupt voltage is at or within +/−0.1 mV of the redox peak current voltage, and further, wherein data collected from the amperometric scan includes data selected from the group consisting of average samples, and composite samples In another embodiment, the present invention involves a method for identifying a concentration of an analyte. The method involves exposing an electrochemical sensor having a diagnostic electrode to the analyte; then scanning the diagnostic electrode using scanning voltammetry; where the scanning voltammetry comprises a square wave voltametric waveform at a first frequency while continuously interrogating current as a function of time. Next, generating a set of readings from the scanning voltammetry. Finally, identifying one or more changes in concentration of the analyte using the set of readings. In another embodiment, the scanning voltammetry is conducted without calibration. In one embodiment, the electrochemical sensor has been calibrated.

In one aspect of the present invention, a method of interrogating and applying an electrochemical aptamer-based (EAB) sensor is disclosed. The method involves applying a square wave voltametric waveform at a first frequency to the EAB sensor while continuously interrogating current as a function of time and collecting the resulting data. In one embodiment, the first frequency is 100 kHz. In another embodiment, a single voltametric sweep is used for simultaneous signal optimization and calibration. In one embodiment, the resulting data is used to determine critical frequency.

In another aspect of the present invention, a method of obtaining the entire frequency response of an electrochemical aptamer-based (EAB) sensor is disclosed. The method involves conducting a square wave voltammetric sweep at a first frequency to the EAB sensor while continuously interrogating current as a function of time without a target and then conducting a square wave voltammetric sweep at the first frequency to the EAB sensor while continuously interrogating current as a function of time with a saturated target condition.

In another aspect of the present invention, a method for identifying a concentration of an analyte is disclosed. The method involves first exposing an electrochemical sensor having a diagnostic electrode to the analyte. Next, scanning the diagnostic electrode using scanning voltammetry. The scanning voltammetry comprises a square wave voltametric waveform at a first frequency while continuously interrogating current as a function of time. A set of readings is generated from the scanning voltammetry. One or more peaks are identified in the set of readings. The concentration of the analyte is determined by applying a predetermined correlation to a voltage difference between the identified peak(s). In one embodiment, the first frequency is 100 kHz. In another embodiment, a single voltametric sweep is used for simultaneous signal optimization and calibration. In one embodiment, the resulting data is used to determine critical frequency.

### BRIEF DESCRIPTION OF THE DRAWINGS

The objects and advantages of the disclosed invention will be further appreciated in light of the following detailed descriptions and drawings in which

FIG. 1A is a graph showing the potential and current vs. time for a traditional SWV with a commercial instrument.

FIG. 1B is a graph showing current vs. potential for nSWV at 100 Hz.

FIG. 1C is a graph showing the potential and current vs. time for the continuous SWV of the present invention.

FIG. 1D is a graph showing current vs. potential for ncSWV at 100 Hz.

FIG. 2A is a graph showing current vs. potential for SWV showing forward, reverse and difference curves.

FIG. 2B is a graph showing current vs. potential for cSWV showing forward, reverse and difference curves.

FIG. 3A is a graph showing peak current vs frequency using SWV for Ferrocene and K₃[Fe(CN)₆].

FIG. 3B is a graph showing peak current vs frequency using cSWV for Ferrocene and K₃[Fe(CN)₆].

FIG. 3C is a graph showing peak current vs frequency using cSWV for Ferrocene and K₃[Fe(CN)₆].

FIG. 4A is a graph showing current vs. potential using SWV for ATP with and without target.

FIG. 4B is a graph showing current vs. potential using cSWV for ATP with and without target.

FIG. 4C is a graph showing current vs. potential using SWV for Tobramycin with and without target.

FIG. 4D is a graph showing current vs. potential using cSWV for Tobramycin with and without target.

FIG. 5A is a graph showing % signal change vs. frequency using cSWV for ATP. The inset is an expanded view of percent signal change at lower frequencies up to 2000 Hz.

FIG. 5B is a graph showing % signal change vs. frequency using cSWV for Tobramycin. The inset is an expanded view of percent signal change at lower frequencies up to 2000 Hz.

FIG. 5C is a graph showing % signal change vs. frequency showing SWV results vs. cSWV for ATP.

FIG. 5D is a graph showing % signal change vs. frequency showing SWV results vs. cSWV for Tobramycin.

FIG. 6A is a graph showing normalized current vs. 1/frequency using cSWV for ATP with and without target.

FIG. 6B is a graph showing normalized current vs. 1/frequency using cSWV for Tobramycin with and without target.

FIG. 6C is a graph showing normalized current vs. 1/frequency using SWV for Tobramycin with and without target.

FIG. 6D is a graph showing normalized current vs. 1/frequency using cSWV for ATP with and without target.

FIG. 7A is a graph showing peak current vs. frequency for K₃[Fe(CN)₆].

FIG. 7B is a graph showing peak current vs. frequency for ferrocene.

FIG. 8A is a graph showing signal change vs. frequency for tobramycin.

FIG. 8B is a graph showing current vs. potential for tobramycin.

### DEFINITIONS

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “electrode” means any material that is electrically conductive such as gold, platinum, nickel, silicon, conductive liquid infused materials such as ionic liquids, PEDOT:PSS, conductive oxides, carbon, boron-doped diamond, nanotubes or nanowire meshes, or other suitable electrically conducting materials.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “polynomial fit” means that the relationship between two variables is a polynomial functional relationship.

### DETAILED DESCRIPTION OF THE INVENTION

The present invention involves the application of an abrupt voltage pulse to an electrode on an EAB sensor and the collection of multiple current vs. time data points to determine concentration of an analyte. The method applies an abrupt voltage pulse to an electrode. This causes redox electron transfer between redox tags on the aptamers and the electrode. After application of that pulse, two or more samples of redox current vs. time are collected and samples are used to calculate sensor response or target concentration. In one embodiment, amperometry is used to interrogate the sensor. In another embodiment, square wave voltammetry is used to interrogate the sensor.

In one embodiment, the present invention involves the development of a single waveform (e.g., single voltametric sweep) that allows for simultaneous signal optimization and calibration for folding-based electrochemical sensors. In one embodiment, the invention involves the use of this new waveform, termed continuous squarewave voltammetry, to interrogate and apply electrochemical, aptamer-based sensors. By continuously collecting current for the entire voltammetric sweep (100 kHz data sampling frequency), information can be extracted that is equivalent to running several voltammograms at various frequencies that range from 50 kHz to the nominal square wave frequency used in the potential waveform.

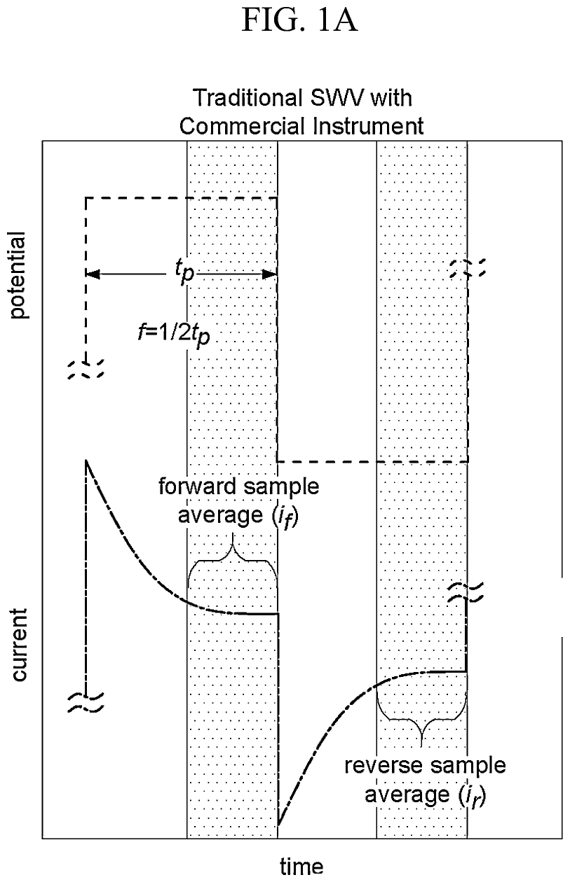

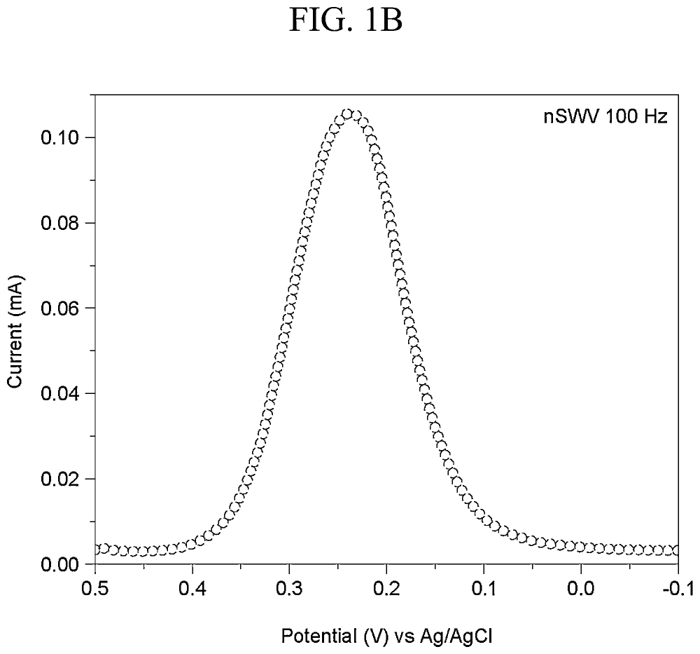

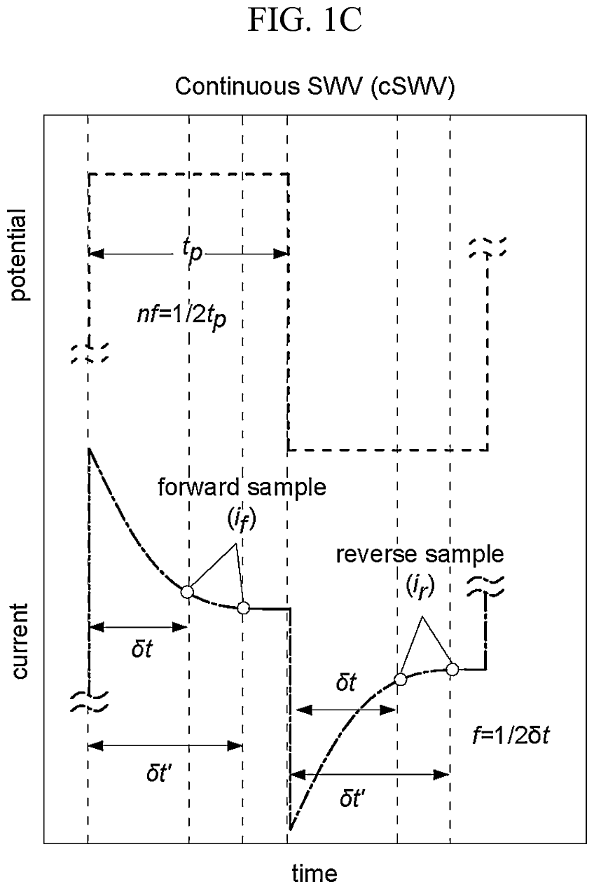

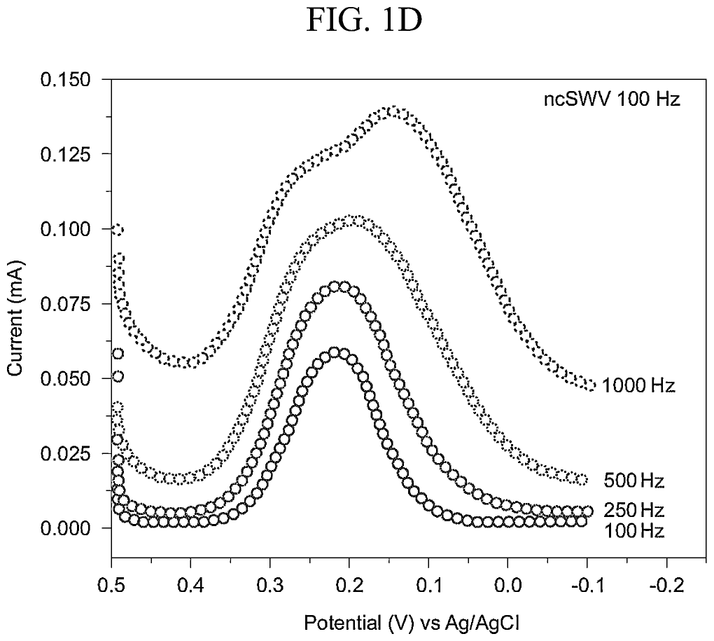

The present invention utilizes the complete current-time response gained during the application of square wave voltammetry waveform to collect continuous square wave voltammograms (cSWV) of both soluble and surface-bound redox markers to maximize the information content obtainable from a single voltammetric sweep. In traditional square wave voltammetry (SWV), the frequency (f) of the square wave potential pulse is defined as 1/(2tₚ), where tₚ is the pulse width (FIG. 1A). Typically, the current is sampled at the end of the forward and reverse pulse, and the difference (i_{f}−iᵣ=i_{diff}) is plotted against potential. In practice, many commercial instruments used here average the current from the latter half of each pulse to determine i_{f} and iᵣ, which provides an average current over that time period. This will provide increased signal-to-noise ratio in the resulting voltammograms. In either case, the majority of the current data obtained during the voltammetry is discarded.

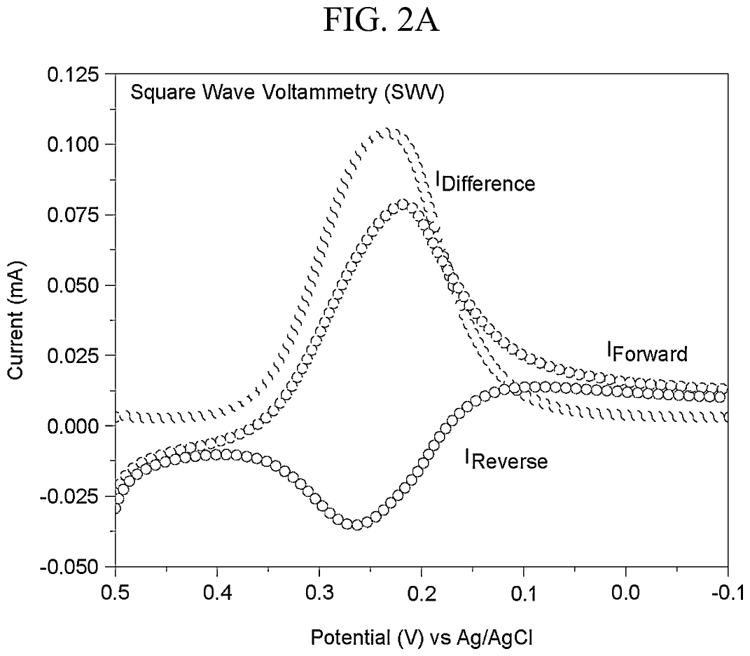

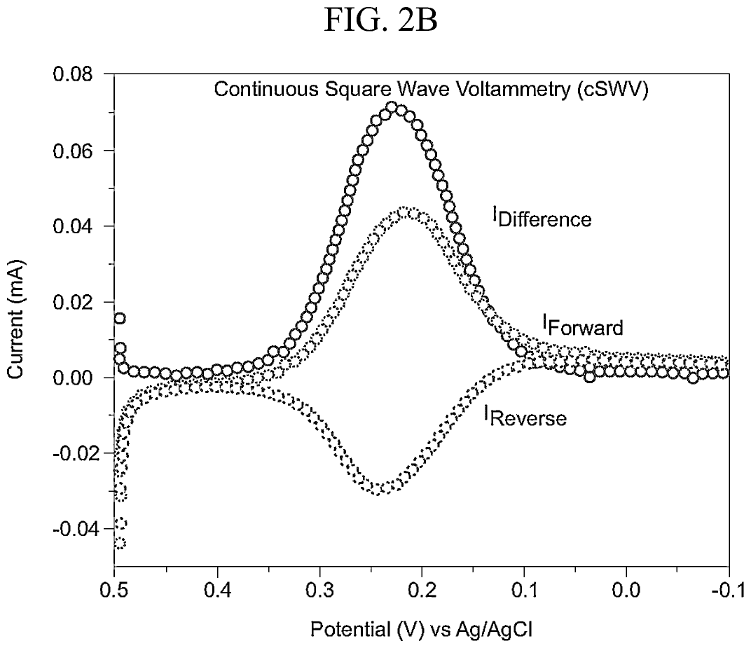

Square wave voltammetry that collects all current time data is defined herein as “continuous square wave voltammetry” (cSWV). To conduct cSWV, a native frequency is selected, which is equivalent to a traditional square wave frequency (f). This native square wave frequency dictates the length of each potential pulse (FIG. 1C). However, during a single native frequency voltammetric sweep, the current is collected at a sampling frequency of 100 kHz. Because of this collection frequency, individual voltammograms can be extracted at effective frequencies as defined below. To better describe cSWV, a number of variables were defined. First, δt (s) is defined as the time after applying individual forward and reverse pulses. cSWV frequency is then calculated as 1/2δt (Hz), where the native frequency is defined as 1/(2δtₘax) or the length of the entire pulse tₚ as defined above. Since data is collected at 100 kHz, a voltammogram taken at a native frequency of 50 Hz can generate 1000 different voltammograms corresponding to 1000 frequencies. (no. of voltammograms=tₚ×sampling frequency). The experiments were performed using a custom LabVIEW code and were run simultaneously with the CH instrument's amperometric trace to collect the entirety of the current time data. The current values corresponding to each voltammogram were plotted against the potential window used to monitor the reaction (FIG. 2A).

Voltametric interrogation applies a standard square wave voltametric waveform at a given frequency while continuously interrogating current as a function of time (and thus by conversion of scan rate to potential, also potential). With continuous current sampling, one can then pick a specific δt, or time after the application of a pulse (forward and reverse), and plot the differential current (i_{f}−iᵣ) at the chosen δt between the forward and reverse pulse. Plotting this differential current vs. time or potential at a given δt yields a sampled current voltammogram at a squarewave frequency of 1/δt. By collecting data continuously, any squarewave frequency between 1/δtₘᵢₙ→∞ and 1/δtₘₐₓ→0 is achievable in a single voltametric sweep. Because folding-based sensor performance (i.e., sensitivity) is a function of squarewave frequency or 1/δt, one can quickly find the optimal frequency for signaling from two voltametric sweeps, one without target analyte present and one with target present.

A consequence of a finite number of redox probes on a surface with different electron transfer rates in the presence and absence of target is there is also a time point at which the currents, with or without presence of target, will be equal (δt_{eq}). With the present continuous squarewave voltammetry waveform, calibration can be achieved with a single voltametric sweep followed by analysis of the current at dt_{eq} and any other δt. This provides a self-referencing point for “on the fly” calibration. These measurements are achievable because in the course of a single voltametric sweep, by collecting current data continuously, you obtain information about each potential frequency achievable.

The continuous square wave voltammetry technique of the present invention collects the entire set of current time data related to a single voltammetric sweep. Unlike square wave voltammetry which provides a single averaged voltammetric response for a faradaic reaction, cSWV inherently provides multiple voltammograms corresponding to frequencies ranging from 50 kHz to the native interrogation frequency, while retaining the benefits of SWV compared to other possible measurement techniques. An important advantage of using cSWV is the ability to perform these same types of measurements in a series of 1 to 2 voltammetric sweeps as opposed to a large number of sweeps. For example, sensor optimization to determine optimal interrogation frequency often requires ˜>20-30 voltammetric sweeps at a wide range of SW frequencies (10-5000 Hz). This can now be achieved with two sweeps, one with target and one without. Moreover, the dual frequency approach disclosed in the art is a calibration free technique where target concentrations are determined regardless of sensor to sensor surface variation and without the need to be recalibrated against the reference sample. This is achieved by voltammetrically interrogating two different SWV frequencies corresponding to a responsive and non-responsive (current independent of target concentration) signal. Another instance where the frequency dependence of EAB sensors employed is explained by the same group where they have shown an improved calibration system for EAB sensor interrogation by tuning the square wave frequency in order to get the maximum peak current responses given at signal on and signal off instances by the same sensor hence determining the “Kinetic differential measurement” (KDM) values. This application enables determination of target concentration with clinical accuracy. The present cSWV technique can be effectively incorporated for many such applications. Some important advantages in each of these two examples is the improved temporal resolution achievable by negating the need to perform multiple scans. A single sweep can provide information at two or more different SWVs for calibration purposes thus improving the rate at which quantitative target concentration data can be collected. First, continuous square wave voltammetry was tested with soluble redox markers ferrocene and K₃[Fe(CN)₆], followed by interrogation with EAB sensors fabricated with ATP and tobramycin aptamers. Similar trends in the variation of the peak current with frequency were observed with both soluble redox markers and EAB sensors. Furthermore, this technique allows the determination of critical frequency, which provides information related to kinetics and sensor behavior.

Regarding FIGS. 1A-1D, continuous squarewave voltammetry (cSWV) employs the same applied potential waveform as what is used in commercial instruments (here CH Instruments) employing square wave voltammetry. FIGS. 1A and 1B show that in traditional square wave voltammetry the frequency (f) of the square wave potential pulse is defined as 1/(2tₚ) where, is the pulse width. Typically, current is sampled at the end of the forward pulse and reverse pulse and the difference (i_{f}− iᵣ=i_{dff}) is plotted against potential. The commercial instrument used here averages the current from the latter half of each pulse to determine i_{f} and iᵣ. FIGS. 1C and 1D show that cSWV continuously collects current data throughout the applied potential waveform at a native SWV frequency of nf=50 Hz with a data collection rate of 100 kHz. Selecting current at a given time after the application of a pulse (δt) allows the extraction of a voltammogram at a frequency=½δt. At nf=50 Hz, and data collection frequency of 100 kHz, allows for the extraction of 1000 voltammograms from a single voltametric sweep.

Regarding FIGS. 2A and 2B, continuous square wave voltammetry (cSWV) provides similar diffusion-limited current-voltage responses for soluble redox markers to what is observed with traditional square wave voltammetry. FIG. 2A shows that traditional SWV for 5 mM K₃[Fe(CN)₆] in 0.1 mM KCl yields typically forward, reverse, and difference voltammograms taken at 100 Hz with an increment of 1 mV and step size of 25 mV. FIG. 2B shows that cSWV voltammograms extracted from a voltammogram at a native frequency of 10 Hz using a δt of 5 ms (½δt=100 Hz) yield equivalent voltammograms at 100 Hz.

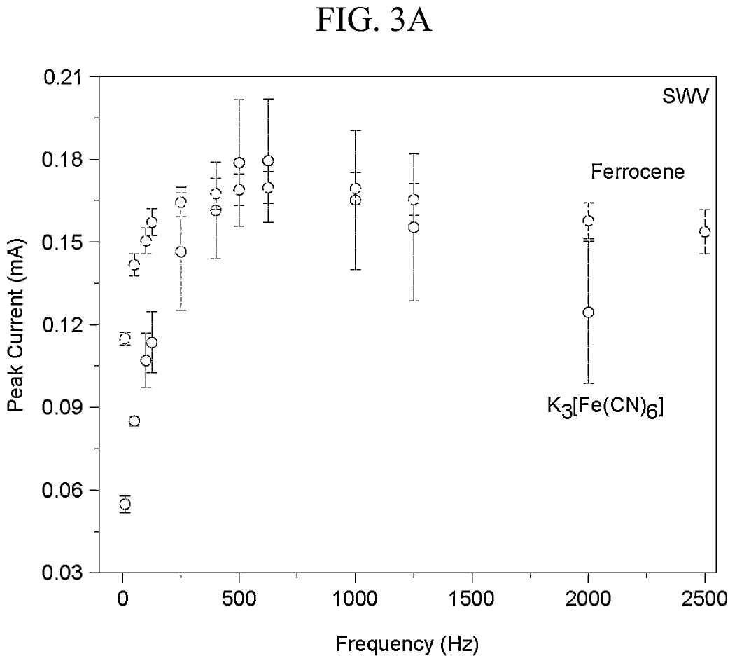

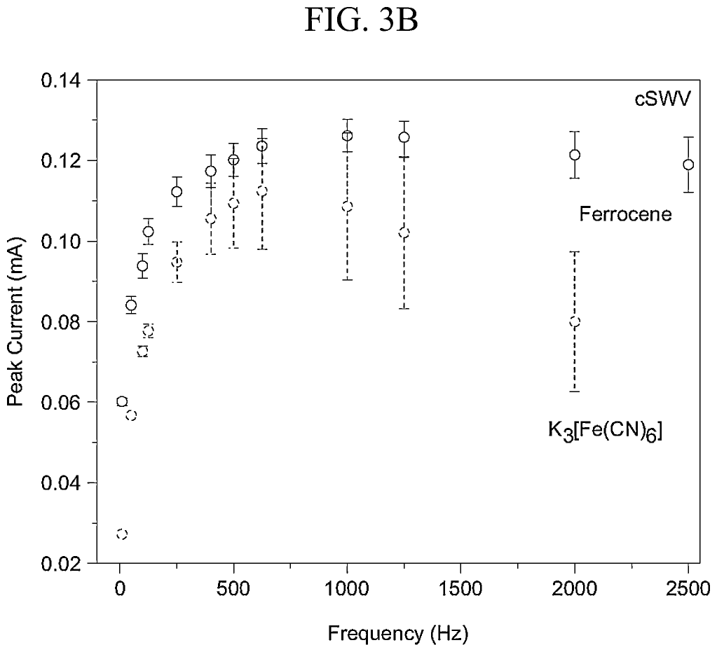

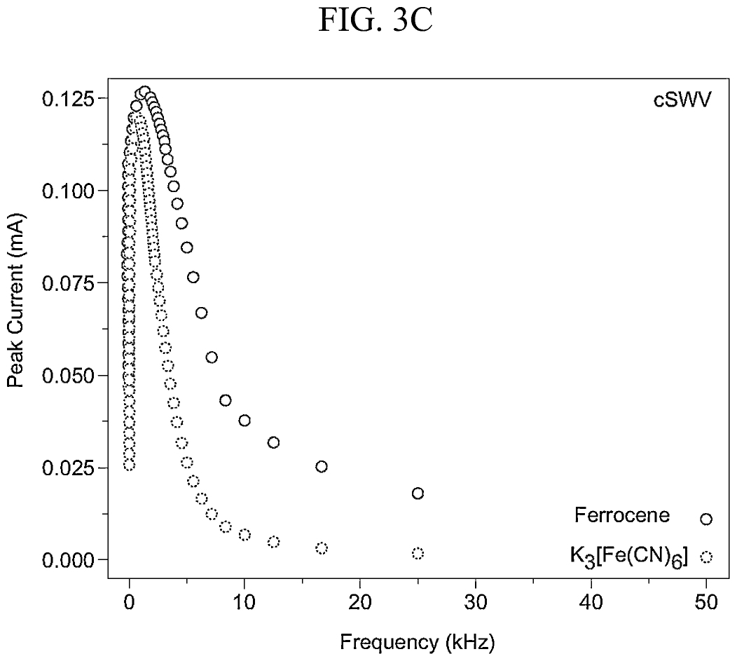

FIGS. 3A-3C show that the peak current dependence on the frequency with the soluble redox marker K₃[Fe(CN)₆] and ferrocene were investigated with both techniques. FIG. 3A shows peak currents analogous to voltammograms acquired with traditional SWV at different frequencies for 5 mM K₃[Fe(CN)₆] in 0.1 mM KCl and 5 mM Ferrocene in 0.1 mM Tetrabutylammonium hexafluorophosphate are shown. SW Voltammograms were obtained by scanning from 0.5 to −0.1V potential window for K₃[Fe(CN)₆] and 0.8 V-0.1 V for ferrocene with frequencies ranging from 10 Hz-2500 Hz. FIG. 3B shows peak current responses extracted from voltammograms obtained at frequencies equivalent to SWV from cSWV run at a native frequency of 10 Hz. FIG. 3C shows all the peak current values that are plotted against the full range of frequency given from cSWV. Solution conditions and parameters used are the same as traditional SWV.

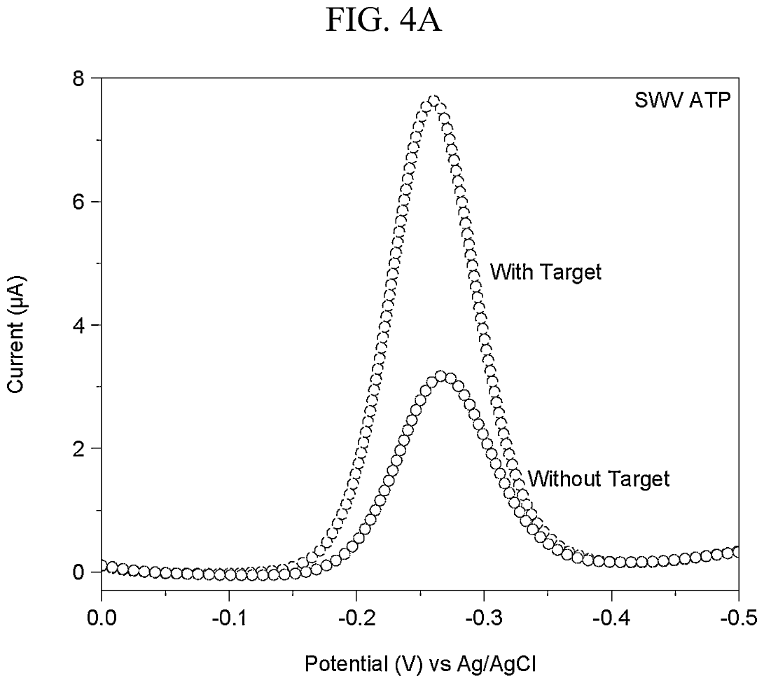

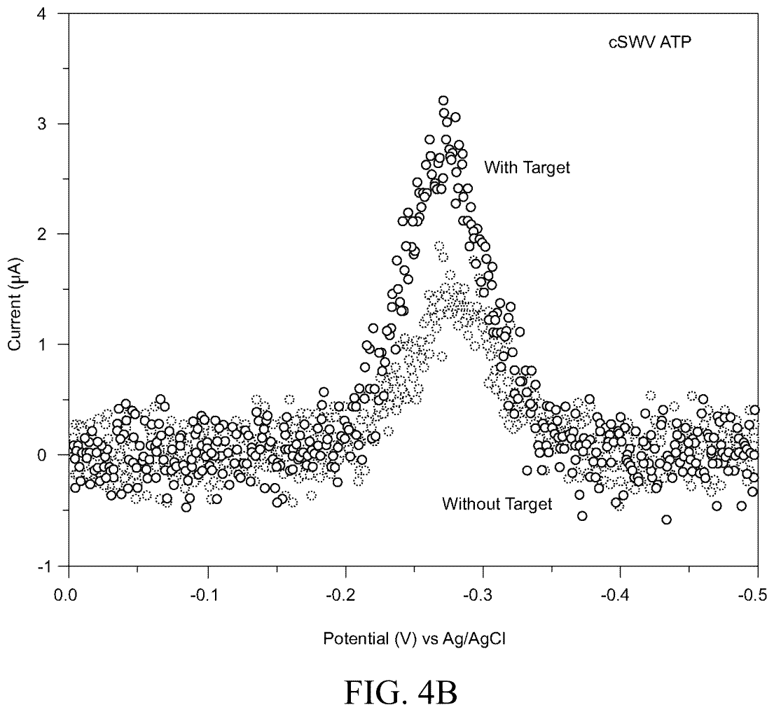

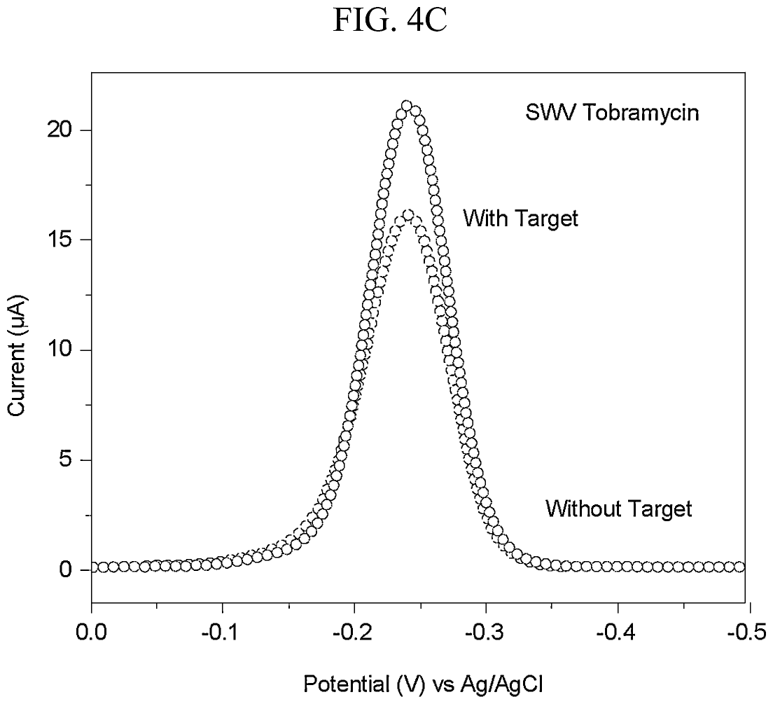

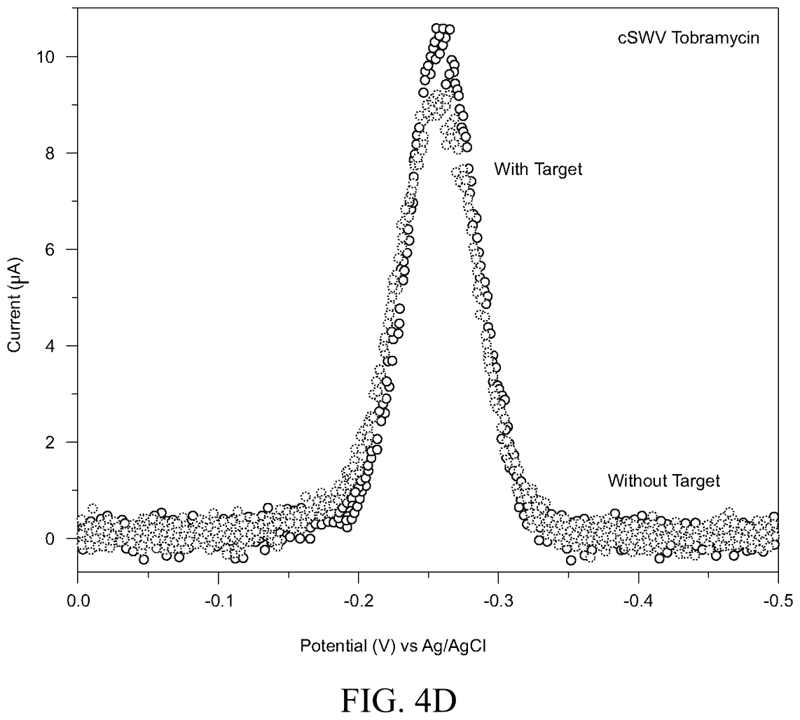

Regarding FIGS. 4A-4D, representation of voltammetric response from the two techniques for bound and unbound states are shown. FIGS. 4A and 4B show voltammograms obtained for ATP sensors interrogated with SWV at 400 Hz for the bound and unbound stage and voltammograms equivalent to 400 Hz (δt=1.25 ms) acquired with cSWV at native frequency 50 Hz under identical conditions as SWV FIGS. 4C and 4D show aminoglycoside tobramycin sensor voltammetric response at 500 Hz upon 1 mM target addition with both techniques. The equivalent voltammogram for 500 Hz with cSWV is obtained at δt=1 ms.

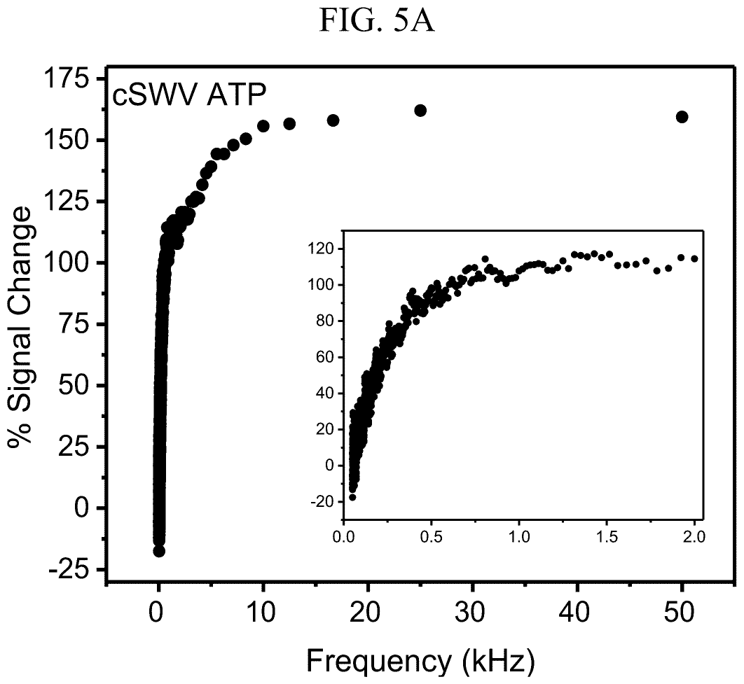

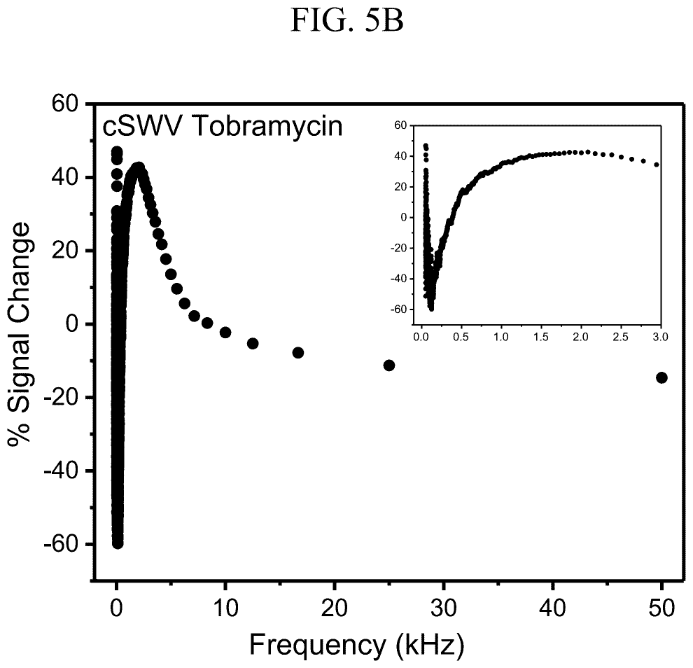

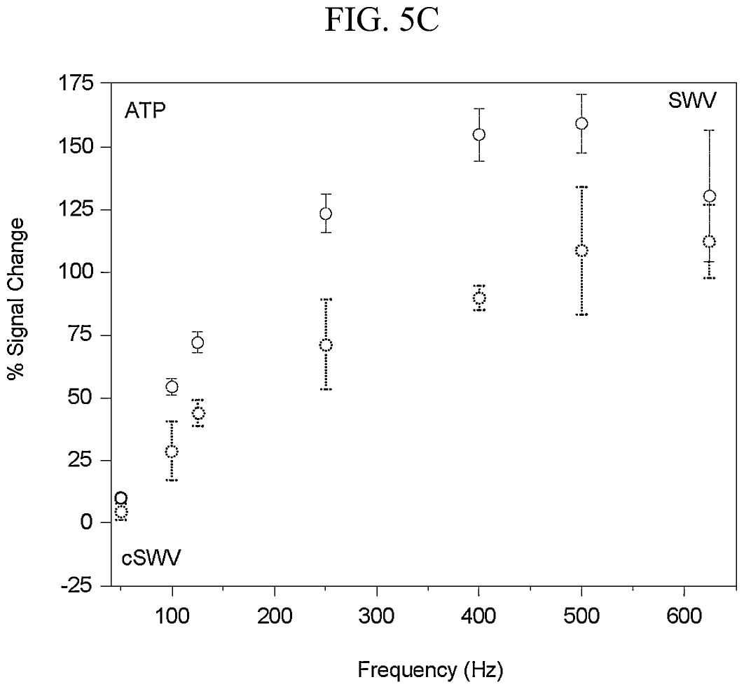

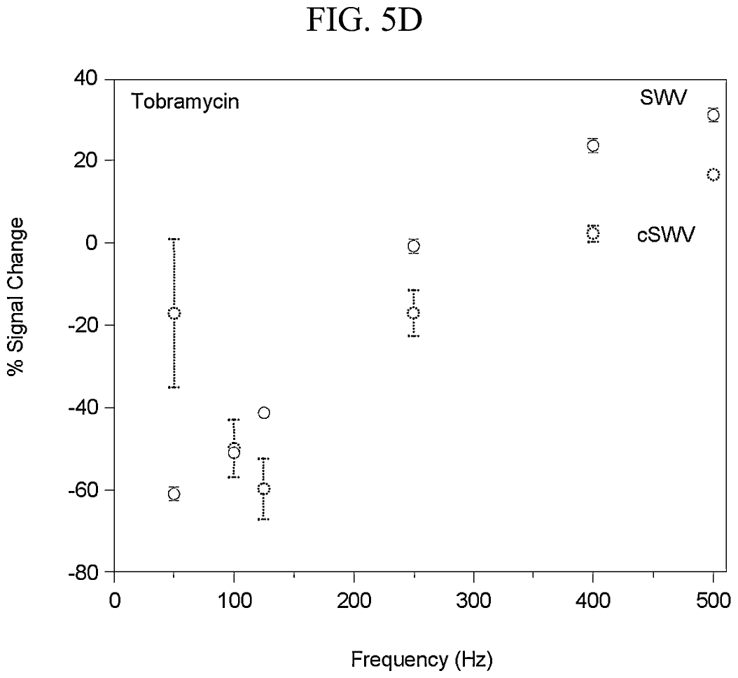

Regarding FIGS. 5A and 5B, calculated percent signal change responses are shown for ATP and tobramycin EAB sensors from SWV and cSWV. All the percent signal change values given by cSWV for the full range of frequencies from 50 Hz to 50 kHz were acquired with ATP and tobramycin sensors respectively. The insets represent an expanded view of percent signal change at lower frequencies up to 2000 Hz. The percent signal change response for ATP and tobramycin sensors were calculated for the frequencies ranging from 50 Hz to 500 Hz with SWV. Their comparison with equivalent frequencies acquired from cSWV of native frequency 50 Hz is shown.

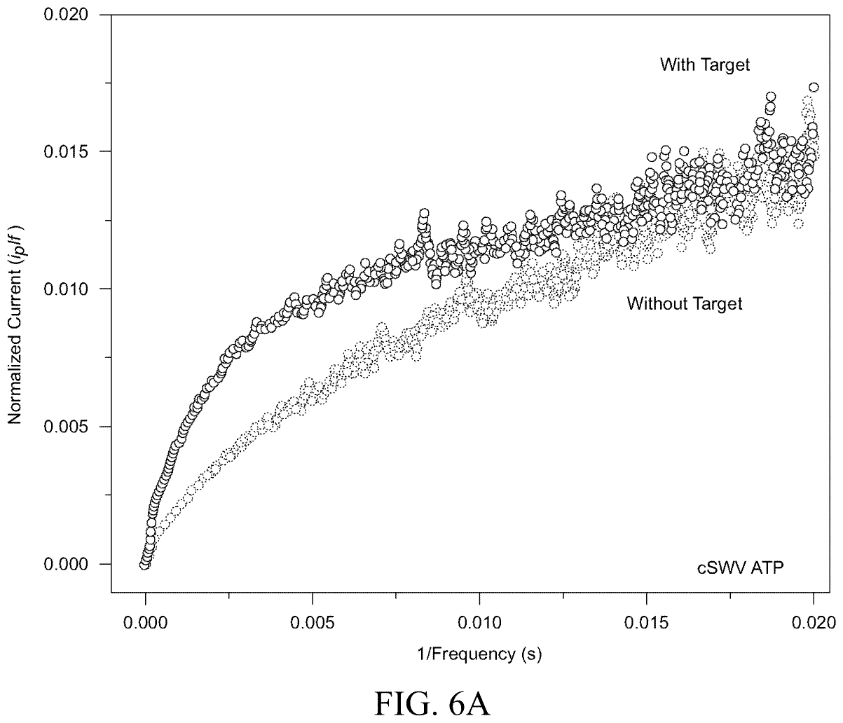

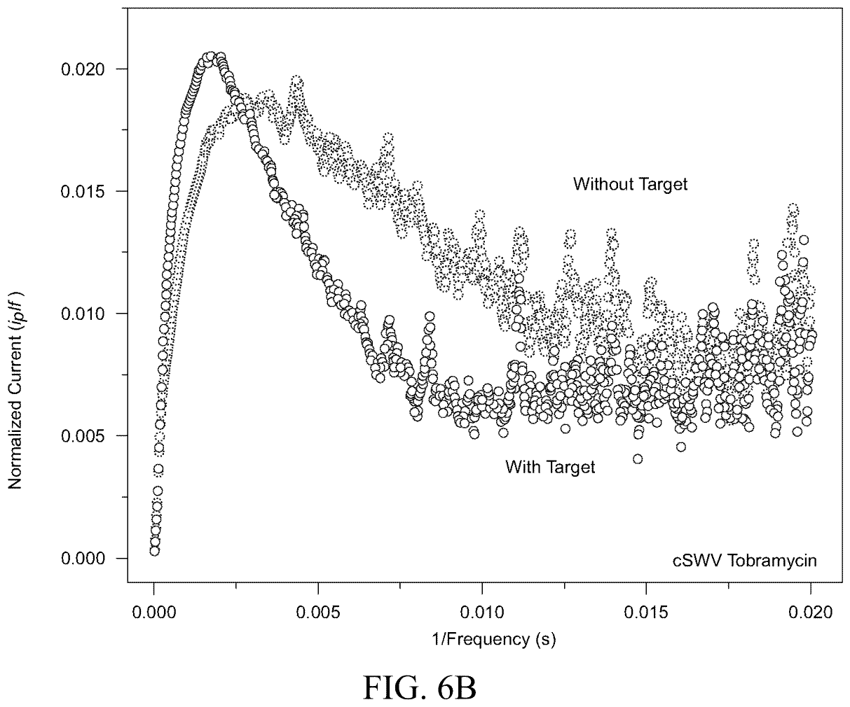

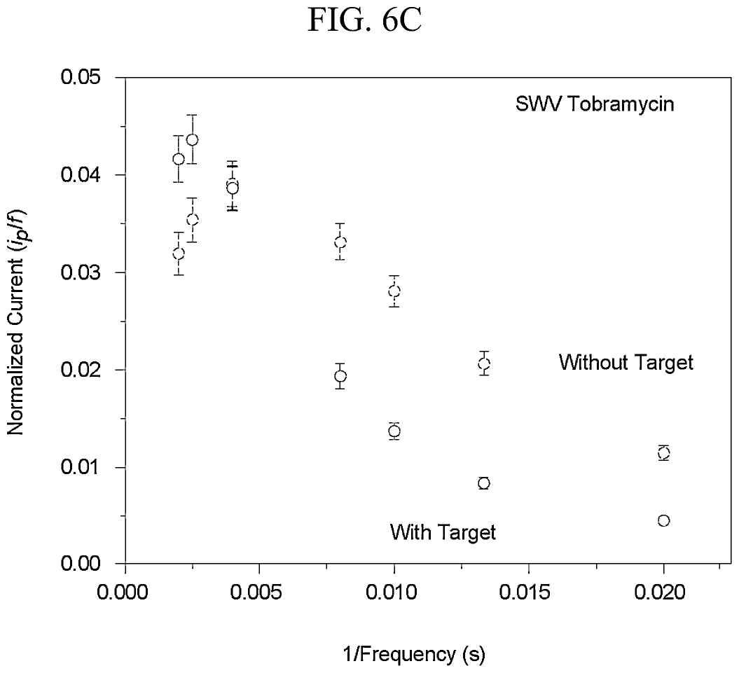

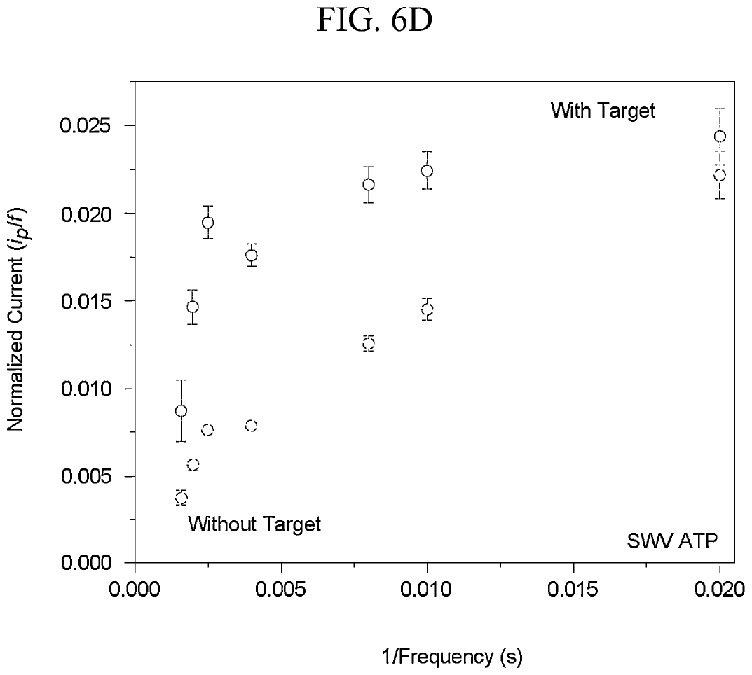

Regarding FIGS. 6A-6D, square wave voltammetry allows determination of electron transfer kinetics between the electrode and the redox probe, and this can be incorporated with the new technique cSWV. FIGS. 6A and 6B show plots of normalized current (iₚ/f) vs 1/frequency (1/f) could provide the apparent electron transfer rates from the plot maximum and are displayed for ATP and tobramycin sensors respectively. To compare the electrode kinetic information obtained from cSWV, FIGS. 6C and 6D show traditional SWV that was performed in a selected range of frequency for the same EAB sensors. Similar trends are observed with the two techniques for the “with target” and “without target” states.

cSWV Response from Soluble Redox Markers

cSWV voltammograms of the soluble redox marker K₃[Fe(CN)₆] are similar to those collected via SWV when using 100 Hz native frequency. For example, the voltammetric response for the forward, reverse, and difference voltammograms of 5 mM K₃[Fe(CN)₆] in 0.1 mM KCl followed the typical response for a soluble redox marker. Both the traditional SWV and cSWV voltammograms were performed at a native frequency of 100 Hz, and in the cSWV case, the voltammogram displayed is at a δtₘₐₓ=tₚ=5 ms. (FIGS. 2A and 2B).

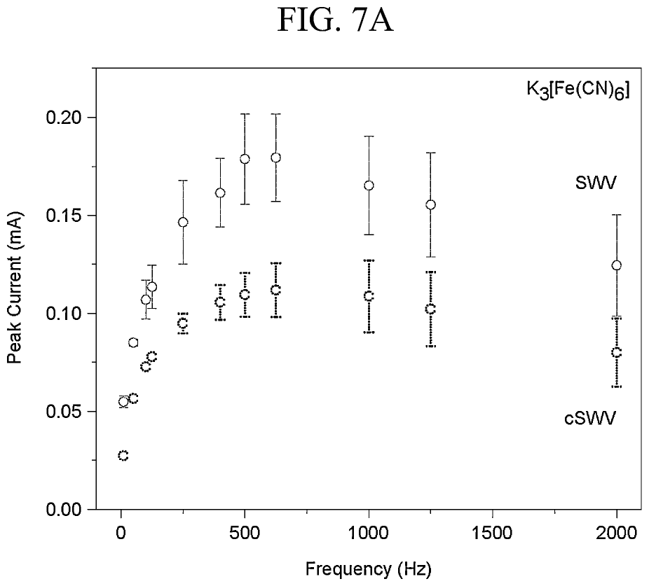

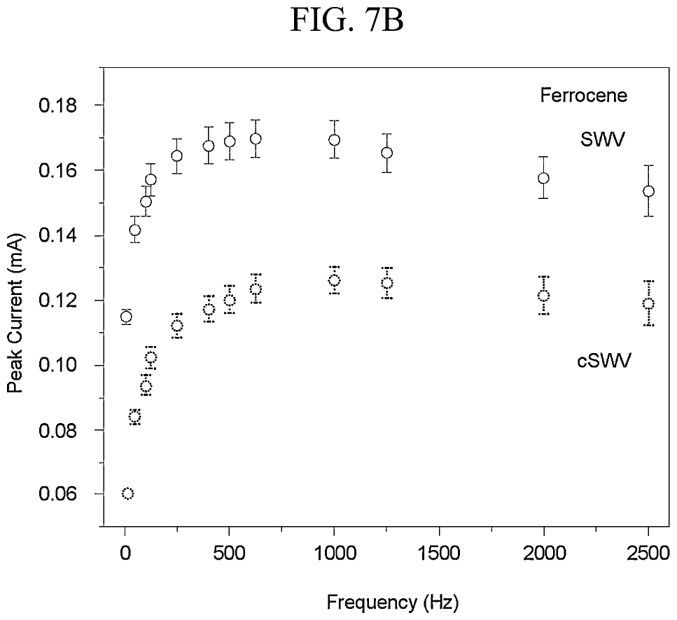

The frequency response, and thus scan rate response, of soluble redox markers, are the same for both traditional SWV and cSWV (FIGS. 3A-3C). When studying K₃[Fe(CN)₆] with 0.1 mM KCl, with traditional square wave voltammetry, the peak current increases monotonically until ˜625 Hz, followed by a decreasing trend in peak current as expected since the reaction behaves in a quasi-reversible way at these higher frequencies because of the finite electron-transfer kinetics. The response of ferrocene with 0.1 mM TBAPF₆, conversely, plateaus at a similar frequency indicating the reaction behaving reversibly even at the highest frequency tested (2500 Hz). Using the same set of electrodes, we find a similar peak current response with cSWV up to 2500 Hz; however, we can probe at much higher frequencies with cSWV. We still find that peak currents begin to roll over or plateau at ˜625 Hz, similar to SWV for K₃[Fe(CN)₆] with 0.1 mM KCl and ferrocene in 0.1 mM TBAPF₆ respectively (FIG. 3B). To better visualize the comparison of peak current responses obtained from traditional SWV to the equivalent frequencies of cSWV performed at a native frequency of 10 Hz are shown in the FIGS. 7A and 7B. More specifically we observe a maximum current response at 957±32 Hz and 1721±162 Hz for K₃[Fe(CN)₆] with 0.1 mM KCl and ferrocene in 0.1 mM TBAPF₆ respectively when all the frequencies from cSWV are analyzed (FIG. 3C).

cSWV to Interrogate Electrochemical, Aptamer-Based (E-AB) Sensors

Square wave voltammetry is a common electrochemical technique used to interrogate electrochemical, aptamer-based (or E-AB) sensors. E-AB sensors are utilized as an analytical detection tool for a wide variety of target molecules ranging from small molecules to proteins. They can even be employed in vivo for continuous therapeutic monitoring. Briefly, E-AB sensors employ nucleic acid aptamers (single-stranded DNA or RNA oligonucleotide strands that bind to targets of interest) that are thiol modified at the 5′ end and attached to the electrode surface (e.g., gold). In contrast, the 3′ end is modified with a redox marker like methylene blue. In the presence of the target, the aptamer undergoes a conformational change bringing the redox tag closer to the electrode surface, facilitating the electron transfer process. SWV is particularly well suited to monitor this class of sensor because of the ability to reduce background currents (non-faradaic charging current) while maximizing signaling differences between the target-free and target-bound states. Prior research reported the optimization of electrochemical signaling via the variation of interrogation frequency as well as a measure of the apparent charge transfer rates with these types of folding-based sensors. More recently, this approach was expanded on using variable frequency interrogation to perform on-the-fly calibration of a sensor and eliminate sensor drift when employed in vivo. As such, measuring the frequency-dependent response of E-AB sensors is critical to the function and application of this powerful class of sensors.

To demonstrate the applicability of cSWV to EAB sensors, we tested the method with two representative sensors fabricated to detect adenosine triphosphate (ATP) and aminoglycoside antibiotics (tobramycin). Qualitatively and quantitatively, E-AB sensors interrogated via SWV and cSWV performed comparably. For example, sensors fabricated with the destabilized ATP aptamer exhibit a 154±10% signal change when interrogated at a SWV frequency of 400 Hz. Similarly, a percent signal change of 90±5% is shown with equivalent 400 Hz SWV frequency (δt=1.25 ms) extracted from 50 Hz native cSWV frequency upon target addition of 1 mM ATP (FIGS. 4A and 4B). Sensors fabricated with the aminoglycoside binding aptamer behaved similarly, exhibiting signal change of 31±1% with SWV at 500 Hz and 17±1% cSWV at δt=1 ms upon addition of 1 mM tobramycin target. (FIGS. 4C and 4D).

An advantage of using cSWV is that the entire frequency response of an E-AB sensor is obtained from simply two voltammetric sweeps—one without a target and one with saturated target condition. Typically, a frequency sweep is obtained by running voltammograms over a range of frequencies in buffer and buffer saturated with target. Accordingly, a typical frequency sweep for the ATP and tobramycin sensors taken with SWV, exhibits an expected frequency-dependent signal change (FIGS. 5A and 5B). To quantify the sensor responses gathered by the two techniques, percent signal change was calculated using the equation (i_{WT}−i_{NT}/i_{NT})×100, where i_{WT} is the peak current response in the presence of the target and i_{NT} is the peak current response with no target. Note that these data required taking 12 voltammograms for 6 frequencies (with and without target). Conversely, two voltammetric sweeps of cSWV taken at a native frequency of 50 Hz with and without target, yields 1000 voltammograms between 50 Hz and 50 kHz (FIGS. 5A and 5B). When the same 6 frequencies are isolated from the native 50 Hz sweep, we find that the signal change upon target addition matches with what is observed with SWV (FIGS. 5C and 5D). Plotting signal changes at every collected δt, and thus frequency gives a highly resolved view of the frequency response of the respective E-AB sensor. The most remarkable percent signal change for ATP sensors is observed at around 175% given at 25000 Hz, and overall higher percent signal changes (>100%) are observed at higher frequencies (>500 Hz). Similarly, tobramycin sensors provided the highest percent signal changes, around 50%, and both signal on and off response was observed as expected from tobramycin sensors. Moreover, they both exhibit frequencies of no response. The key difference here is data (peak currents or signal change) can be acquired at desired frequencies (maximum signal change and frequency of no response) with only a single voltammetric sweep. Alternatively, one can perform an entire frequency sweep to find the optimal signaling with only two voltammetric sweeps. There is a slight discrepancy seen in percent signal changes obtained for ATP and tobramycin EAB sensors. But the two techniques are comparable to each other in calibration performance and follow a matching trend. The dissimilarity in the values between the two techniques could be due to the difference in sampling procedure followed by the two techniques. Averaging of the data in nSWV could result in more gain compared to cSWV and could lead to dissimilarities in the percent signal changes observed. For the tobramycin, EAB sensors much higher error bars are observed with cSWV technique compared to nSWV, especially with 50 HZ and cause a slight deviation from the nSWV response. This may be an artifact of the sensor-to-sensor variability which can impact the overall signal gain by individual sensors.

Another advantage of using cSWV is assessing the charge transfer rates of tethered redox molecules quickly and accurately with high resolution. CSWV allows the determination of critical frequency, as shown in FIGS. 6A and 6B. The critical frequency is given by the maximum of the iₚ/f vs 1/f, where iₚ is the peak current and f is the frequency. The charge transfer rate for the bound and the unbound probes for tobramycin fabricated sensors are determined to be at 480±14 s⁻¹ and 251±37 s⁻¹ respectively with the cSWV technique. Similar observations were noted with SWV where the charge transfer rates for the bound state and the unbound state are 400±150 s⁻¹ and 250±150 s⁻¹ respectively. However, when using cSWV, the frequency resolution is much higher (100 kHz sampling rate leads to a voltammograms at every 1/20 μs frequency) compared to the resolution using nSWV, which is based on how many sweeps the experimenter performs. For example, in our nSWV determination for tobramycin, we find a charge transfer rate of 400 s⁻¹. This determination is based on a max current value observed at 400 Hz flanked by a measurement at 500 Hz and 250 Hz thus the charge transfer rate can only be known within a +/−150 Hz range using this spacing on nSWV. Both techniques provide comparable charge transfer rates for the tobramycin sensor assuring that cSWV technique could be of many advantages to the EAB sensor field. Similar to the observations with the tobramycin sensors, comparable trend in the plots for ATP sensors are observed further confirming the capabilities of the technique. According to the trend given in the plot for ATP, a maximum could not be determined since the plots depicts an increasing trend with 1/f Therefore, a critical frequency value for the with and without target states of the ATP sensor is undetermined.

Another advantage of cSWV is that it can also capture the benefits of forward sample and reverse sample averaging for nSWV. For example while in FIG. 1C samples δt and δt′ are represented as a single datapoint capture, any capture point δt with cSWV can also be an averaging of adjacent captured points and satisfy the principles of the present invention as long as: (1) two or more samples δt and t′ (and possibly δt″ etc.) are captured off a single cSWV, (2) the two or more samples are used in conjunction to provide the sensor response or to calibrate the sensor. For example, if the sampling was at 50 kHz, sample δt or t′ could be 100 μs in width of sampling and each containing 5 current data points each taken at 20 μs intervals. These samples could be averaged into a mean (or other statistical technique, such as median, or other) into a composite value for δt or t′ that is therefore more accurate or precise in measurement. Based on the time-scale of the electron transfer kinetics and the width of the sample for δt or t′ the averaging could introduce error due to the non-linearity of the electron transfer kinetics. Therefore, the δt or δt′ can be non-centered such that, for example, if data was collected at 1.26, 1.28, 1.30, 1.32, and 1.34 ms, the average would be used to represent a sample at 1.32 ms even though 1.30 ms is the median because of overdue influence on the averaging by the 1.26 ms sample. Therefore, one embodiment of the present invention involves a sample that is an average of two or more adjacent samples, or a sample that is a composite of two or more adjacent samples.

Another advantage of cSWV is that with a single scan for sensors with both signal ON (add analyte and redox current increases) and signal OFF frequencies (add analyte and redox current decreases) there is a zero-gain frequency where the sensor is non-responsive to analyte. In this case, cCSV can capture data at two or more points, including a signal ON sample δt_{ON} where redox tag current increases with increasing target concentration, a non-responsive sample δt_{NR}, and a signal OFF sample δt_{OFF} where redox tage current decreases with increasing target concentration, two of which or all of which together can increase accuracy of calibration. In this case, the present invention may also capture a plurality of samples, the plurality of samples being at redox current minimum or maximum or other responses to analyte. The samples may even exceed 3 samples, since multiple samples are inherently provided in the cSWV data.

Another advantage of cSWV is that it does not need to be used continuously and can be used interchangeably with nSWV to maximize sensor accuracy and precision over time. For example, a 48 hour sensor may be factory calibrated (calibrated during manufacturer) and use nSWV for multiple measurements. In addition, at 12, 24, and 36 hours of operation cSWV is performed and used to calibrate the nSWV measurements using data post-processing or software, for example. Therefore, the present invention may include at least one cSWV scan and a plurality of nSWV scans.

Another advantage of cSWV is that in some cases it can be calibrated in-vivo. For example, the sensor binding affinity can be tuned such that the sensor primarily captures the medium and high concentrations for a drug such as an anticoagulant drug such as rivaroxaban which is all the information needed to prevent stroke (ensure patient is in the medium concentration range) and bleeding (ensure the patient is not in the high concentration range). In between doses the drug concentration measured by the sensor is close to zero as the drug metabolizes (e.g. rivaroxaban 1-2 hours before the once daily dose). Therefore, this can be assumed to be a zero-concentration calibration point even though drug concentration in the body is not zero. This assumes, for example, less than 2, 5, 10 or 20% of the aptamers are bound to drug. In one embodiment, the present invention includes a method wherein the sensor is calibrated during in-vivo use at known points where concentration of the target analyte is, in different embodiments, less than 2, 5, 10 or 20%. Similar opportunities could exist, for example, for dialysis patients and kidney biomarkers. In one embodiment, the sensor is a 2 week wearable sensor and patients receive dialysis 3 times a week and within 2 hours after dialysis the sensor is calibrated.

In yet another embodiment of the present invention, cSWV techniques can be applied into a ‘continuous chronoamperometry or amperometry’ or cA technique. A voltage can be abruptly applied to the sensor, an amperometric scan is collected which normally would be analyzed for redox current decay time or a monoexponential fit to the redox current decay curve, where the time or fit is used as a way to measure concentration of the target analyte. In the present invention, the amperometric scan may be captured, for example, with a −0.35V voltage abruptly applied, and data selected from the group consisting of δt samples, δt′ samples, δt″ samples, average samples, and composite samples, collected as taught herein.

Numerous techniques for calibration free operation have been reported. All such ‘calibration free’ methods leverage fundamental aspects of electron transfer rates in EAB sensors, and are simply a specific way of doing so with their own advantages and disadvantages (e.g. two-frequency calibration free, chronoamperometric, measuring the redox peak shifts, etc.). The knowledge of these fundamental behaviors of aptamer sensors (e.g. White and Plaxco, Anal. Chem. 2010, 82, 1, 73-76), to calibrating to a reference sensor which does not bind to target, or calibrating to a sensor with zero concentration (which is also a reference sensor effectively), and their exploit for improving sensor accuracy has been known for many years. The present invention likewise may exploit these fundamental aspects of electron transfer rates, with several non-limiting examples now presented.

In one embodiment, the present invention can use a calibration-free method to calculate concentration. Here, cSWV leverages recording the E-AB sensor's output at “responsive” and “non-responsive” square-wave frequencies without the need to do multiple scans as cSWV captures all frequencies in a single scan. The latter are frequencies where the E-AB sensor's signaling is zero or negligible at all possible target concentrations. The resulting non-responsive signal can then be used to calibrate EAB sensors considering it is constant. Furthermore, the use of a calibration-free constant can be used, a, obtained by running a “calibration” set in which square-wave voltammogram peak currents are sampled at the responsive, i, and at the non-responsive, i_{NR}, square-wave frequencies. The peak currents measured in this calibration set are employed to determine the calibration parameters KD, γ and α. With those parameters at hand, the concentration of target can be directly determined via Equation 1:

$$[ T ] = {K}_{D} \frac{i - α {i}_{NR}}{γα {i}_{NR} - i} = {K}_{D} \frac{\frac{i}{{i}_{NR}} - α}{γα - \frac{i}{{i}_{NR}}} \qquad Equation 1$$

wherein [T] is the concentration of the target, K_{D} is the target's dissociation constant, i is a constant comprising the peak current, α is a constant comprising the ratio of output signal at the minimally frequency and target-free output signal, i_{NR} is output current, and γ is a constant comprising the ratio of target-saturated output signal to target-free output signal.

In one embodiment, the collected samples comprise a first sample and a second sample. The first sample has a redox tag current that is minimally responsive to a change in concentration in target and the second sample has a response to change in concentration in target. The current of these two samples is used to calculate concentration of the target.

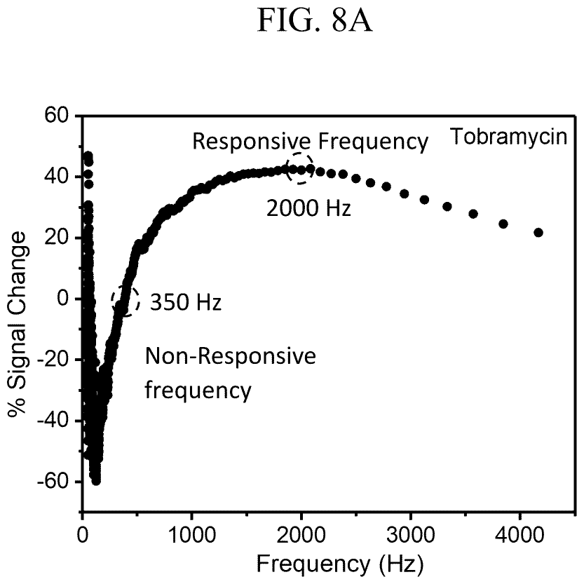

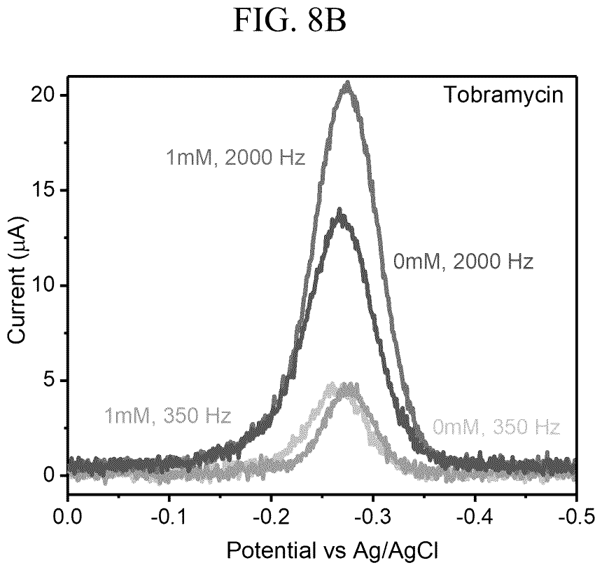

FIG. 8A shows an example of calibration-free analysis using the data from a single sweep of tobramycin. The sweep shows a non-responsive frequency at 350 Hz and a responsive frequency at 2000 Hz. FIG. 8B shows another example, where the lower gray peaks for 1 mM tobramycin and 0 concentration tobramycin are very similar at 350 Hz, but the peaks at 2000 Hz are significantly different.

### Kinetic Differential Measurements (KDM)

In another embodiment, the present invention may use the difference between signal ON and OFF curves to calibrate the E-AB sensor. This approach is called Kinetic Differential Measurements (KDM) and it can be expressed in equation form as:

$${i}_{KDM} = \frac{{i}_{ON}}{{i}_{m i n , ON}} - \frac{{i}_{OFF}}{{i}_{m i n , OFF}} \qquad Equation 2$$

where i_{min,ON} and i_{ON} represent the peak currents measured at the signal-ON frequency in the absence and presence of target, respectively, and i_{min,OFF} and i_{OFF} represent the equivalent measurements performed at the signal-OFF frequency; (4) perform a non-linear regression analysis of the iKDM calibration curve against a binding isotherm; here, for example, using the Langmuir-Hill isotherm:

$${i}_{KDM} = γ \frac{{[ T ]}^{n}}{{K}_{D} + {[ T ]}^{n}} \qquad Equation 3$$

where n is Hill's coefficient (i.e., the number of binding sites on the aptamer). Equation 3 can be used to estimate the dissociation constant (K_{D}) and signal gain (g) of the E-AB sensors. Finally, from knowledge of these parameters and of iₘᵢₙ, target concentration can be derived from measurements of the E-AB sensor's iKDM using:

$$[ T ] = {K}_{D} \frac{{i}_{KDM} - {i}_{m i n}}{γ {i}_{m i n} - {i}_{KDM}} \qquad Equation 4$$

In one embodiment of the present invention, the collected samples have at least a first sample and a second sample. The first sample has a redox tag current that increases with increase in concentration in target. The second sample has a redox tag current that decreases with increase in concentration in target. The difference in changes in current between these two samples is recorded as a differential current value.

### EXAMPLES

### Chemicals and Solutions

Potassium ferricyanide (K₃[Fe(CN)₆]), ferrocene (Fe(C₅H₅)₂), Tris-2-carboxyethyl-phosphine (TCEP), 6-mercapto-1-hexanol, Trizma (tris) base (2-amino-2-hydroxymethyl-1,3-propanethiol), adenosine triphosphate (ATP), magnesium chloride (MgCl₂), tobramycin, 10× Tris-EDTA, tetrabutylammonium hexafluorophosphate (TBAPF₆) were purchased from Sigma-Aldrich (St. Louis, MO, USA) and were used as received. Potassium chloride (KCl), sodium hydroxide (NaOH), hydrochloric acid (HCl), sodium chloride (NaCl), sulfuric acid (H₂SO₄), acetonitrile (C₂H₃N) were purchased from Thermo-Fisher Scientific (Ward Hill, MA, USA) and were used as received. Solutions were prepared with ultrapure water (18.0 MΩ cm at 25° C.) using a Biopak Polisher Millipore ultrapurification system (Millipore, Billerica, MA). Parent tobramycin sequence: 5′-HS-C6-SEQ ID NO:1-MB-3′, destabilized ATP sequence: 5′-HS-C6-SEQ ID NO:2-MB-3′. DNA aptamer sequences were dual HPLC purified (Thermo-Fisher) and were used as received.

| SEQ ID NO: 1 | |
|---|---|
| | GGGACTTGGTTTAGGTAATGAGTCCC |
| SEQ ID NO: 2 | |
| | CTGGGGGAGTATTGCGGAGGAAA |

### Example 1: Electrode Preparation for Soluble Redox Marker Experiments

Polycrystalline gold electrodes of 2 mm diameter (CH Instruments, Austin, TX, U.S.A) were hand-polished with 1 μm diamond suspension solution on a microcloth (Buehler) for two minutes following a figure-eight motion. Similarly, the electrodes were then polished in ultrapure water (Milli-Q Ultrapure Water Purification, Millipore, Billerica, MA, U.S.A) in the same manner. Hand-polished electrodes were sonicated with ultrapure water for 5 minutes and were electrochemically cleaned in different concentrations of sodium hydroxide and sulfuric acid solutions as discussed. The electrodes were cycled in 0.5 M NaOH solution to reductively desorb any attached sulfur molecules to the gold surface. Then oxidation and reduction reactions were performed in 0.5 M H₂SO₄ which aids oxidation of any organic compounds or other available contaminants attached to the gold surface. The surface is then etched with 0.1 M H₂SO₄/0.01 M KCl for further cleaning. These electrochemically cleaned bare electrodes were then interrogated with SWV and cSWV. Soluble redox marker solutions were prepared as follows: 5 mM K₃[Fe(CN)₆] in 0.1 mM KCl in DI water and 5 mM ferrocene in 0.1 mM TBAPF₆ in acetonitrile.

### Example 2: Fabrication of E-AB Sensors

First, 1 μl of aptamer solution was incubated with 2 μl of 100 mM TCEP for 1 hour to reduce the disulfide bonds of the aptamer sequences. Then the electrodes were incubated in a probe solution of 200 nM prepared with 20 mM Trizma Base, 100 mM NaCl and 5 mM MgCl₂ at a pH of 7.40 for 1 hour in room temperature. Then the electrodes were washed well with ultrapure water to remove any excess aptamer and then incubated in 30 mM 6-mercapto-1-hexanol prepared in ultrapure water for passivation. Finally, the well rinsed electrodes were incubated in tris buffer for 1 hour of equilibration

### Example 3: Electrochemical Measurements

All electrochemical measurements were performed with CH instruments 660E and 1040C workstations (CH Instruments, Austin, TX, USA). All electrochemical measurements were collected using a three-electrode cell system consisting of platinum counter electrode, Ag/AgCl (3 M NaCl) reference electrode and 2 mm polycrystalline gold electrodes as working electrodes. Voltammetry parameters are as follows: amplitude and increment were 25 mV and 1 mV unless otherwise noted. The potential window between 0 V to (−0.5 V) and frequencies ranging from 50 Hz to 625 Hz were used for the interrogation of E-AB sensors. Soluble redox markers ferrocene and K₃(Fe[CN]₆) were interrogated at the potential windows between 0.8 V-0.1 V and 0.5 V-(−0.1 V) respectively. They were tested at frequencies ranging from 10 Hz-2500 Hz with SWV. Same parameters were used with continuous square wave measurements and native frequencies of 10 Hz and 50 Hz were selected for soluble redox marker measurements and E-AB sensor measurements respectively, unless otherwise noted. All experiments were conducted with at least three or more electrodes and their standard deviations are represented as error bars.

### Example 4: Continuous Square Wave Voltammetry

Continuous square wave voltammetry was achieved via utilization of the potentiostat capabilities of a CH Instruments (Model 660E) and the potential driving and measurement capabilities of LabVIEW. Briefly, in-house written LabVIEW code (see cSWV.vi in Supporting Information) was developed to generate the square wave voltammetric waveform and apply the voltage waveform through the serial voltage input port on the CH instrument. The serial port on the back of the CH Instruments potentiostat (Model 660E) have the following assignments: 9-Pin D connection; Pin 1-Current 1 Output; Pin 2-Current 2 (bipotentiostat); Pin 3-Inverted Potential Output Pin 4-External Potential Input; Pin 5-External Signal Input; Pins 6-9-Ground. Note that Pin 4 is intentionally disabled as default to avoid instrument noise so it must be enabled to accept inputs via a jumper to connect the proper pins (communication with CH Instruments). Simultaneously, triggered via an external voltage trigger, the output voltage from the serial port on the CH instrument (corresponding to the measured current) is collected via LabVIEW all through a NI USB-6251 data acquisition board at 100 kHz and stored as a .TDMS file. In order to properly interface NI USB-6251 with the CH Instruments, the instrument was run in the chronoamperometric mode in order to turn the electrochemical cell on. Finally, all software filters were disabled prior to the measurements

### Example 5: Data Analysis

The data analysis for the collected cSWV data was done by using MATLAB R2021b as explained. First, the collected data were categorized to the relevant time increments (δt, explained later in text) that correspond to each voltammogram. The original obtained data were smoothed by performing moving mean averaging technique to reduce the noise and facilitate integration of the voltammograms to obtain peak current values. Voltammograms obtained for ATP were noisier compared to the other voltammograms collected via cSWV. Therefore, an additional step of box car averaging was performed to get smoother data that facilitated peak integration.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A method of measuring sensor response for a sample comprising:
a. exposing the sample to an electrochemical aptamer-based (EAB) sensor, wherein the sensor comprises an electrode and one or more aptamers having redox tags;
b. performing a first interrogation comprising applying an abrupt voltage pulse to the electrode, wherein the abrupt voltage pulse causes redox electron transfer between the redox tags on the aptamers and the electrode;
c. collecting two or more data samples at different time values, wherein each data sample is a redox tag current value; and
d. identifying at least one measure of the sample using the data samples.

2. The method of claim 1 wherein square wave voltammetry is used to interrogate the sensor.

3. The method of claim 1 wherein amperometry is used to interrogate the sensor.

4. The method of claim 1 wherein electron transfer rate data is collected after at least one time period selected from the group consisting of 10 μs, 100 μs, and 1 mS.

5. The method of claim 1 wherein sampling is performed at a frequency that is selected from the group consisting of at least 10 Hz, 100 Hz, 1 kHz, 10 kHz and 100 kHz.

6. The method of claim 1 wherein interrogating is conducted without calibration of the sensor.

7. The method of claim 1 wherein the sensor has been calibrated.

8. The method of claim 7 wherein calibration is conducted during manufacture of the sensor.

9. The method of claim 7 wherein calibration of the sensor is performed before use of the sensor.

10. The method of claim 1 wherein the interrogating is conducted twice, wherein one interrogation is done with a target and another interrogation has no target.

11. The method of claim 1, wherein the two or more samples comprise:
a. a minimally responsive sample wherein redox tag current is minimally responsive to a change in concentration of a target, and
b. a responsive sample which has a response to a change in concentration of the target;
wherein current measurements of the two or more samples are used to calibrate the sensor.

12. The method of claim 11 wherein calibration of the sensor is calculated, at least in part, by comparing current measurements of the minimally responsive sample and the responsive sample at the time of response at saturation level through a polynomial fit.

13. The method of 11 where the calibration provides a concentration value, the concentration value calculated according to:
$$[ T ] = {K}_{D} \frac{i - α {i}_{NR}}{γα {i}_{NR} - i} = {K}_{D} \frac{\frac{i}{{i}_{NR}} - α}{γα - \frac{i}{{i}_{NR}}}$$
wherein [T] is the concentration of the target, K_{D} is the target's dissociation constant, i is a constant comprising the peak current, α is a constant comprising the ratio of output signal at the minimally frequency and target-free output signal, i_{NR} is output current, and γ is a constant comprising the ratio of target-saturated output signal to target-free output signal.

14. The method of claim 1, wherein the two or more samples comprise a first sample and a second sample, and wherein the first sample has a redox tag current that increases with increase in concentration in target and the second sample has a redox tag current that decreases with increase in concentration in target, and the difference in changes in current between these two samples is recorded as a differential current value.

15. The method of claim 1 wherein the two or more samples are an average sample averaged from two or more adjacent samples.

16. The method of claim 1 wherein the two or more samples are a composite sample obtained from two or more adjacent samples.

17. The method of claim 1 wherein at least one sample is taken at a time point equivalent to which the change in redox tag current has the largest increase for a given increase in target concentration.

18. The method of claim 1 wherein at least one sample is taken at a time point equivalent to which the change in redox tag current has the largest decrease for a given increase in target concentration.

19. The method of claim 1 wherein at least one sample is taken at a time point equivalent to which the change in redox tag current has least change for a given increase in target concentration.

20. The method of claim 1 wherein the method is a continuous square wave voltammetry scan, and the sensor is measured over time using at least one continuous square wave voltammetry scan for calibration and a plurality of non-continuous square wave voltammatery scans for measurement.

21. The method of claim 1 wherein the sensor is calibrated during in-vivo use at known points where concentration of the target analyte bound to aptamers is less than a percentage selected from the group consisting of 2, 5, 10 and 20%.

22. The method of claim 1 wherein the data samples are used to determine critical frequency.

23. The method of claim 1 wherein the applied abrupt voltage is at or within +/−0.1 mV of the redox peak current voltage, and further, wherein data collected from the amperometric scan includes data selected from the group consisting of average samples, and composite samples.

24. A method for identifying a concentration of an analyte, the method comprising:
a. exposing an electrochemical sensor having a diagnostic electrode to the analyte;
b. scanning the diagnostic electrode using scanning voltammetry; wherein the scanning voltammetry comprises a square wave voltametric waveform at a first frequency while continuously interrogating current as a function of time;
c. generating a set of readings from the scanning voltammetry; and
d. identifying one or more changes in concentration of the analyte using the set of readings.

25. The method of claim 24 wherein the scanning voltammetry is conducted without calibration.

26. The method of claim 24 wherein the electrochemical sensor has been calibrated.


SEQUENCE LISTING (2 sequences)

SEQ ID NO: 1 - DNA, 26 bp, synthetic construct
  source 1..26: mol_type = other DNA
gggacttggtttaggtaatgagtccc

SEQ ID NO: 2 - DNA, 23 bp, synthetic construct
  source 1..23: mol_type = other DNA
ctgggggagtattgcggaggaaa